(19) United States
(12) Patent Application Publication
(10) Pub. No.: US 2007/0148112 A1
(43) Pub. Date: Jun. 28, 2007

(54) Foaming, color-changing topical composition and method of imparting a cosmetic effect

(75) Inventors: Ajay G. Dingley, New Windsor, NY (US); Shari Martin, Suffern, NY (US); Glen T. Anderson, Pleasantville, NY (US)

Correspondence Address: Charles N. J. Ruggiero;Ohlandt, Greeley, Ruggiero & Perle, L.L.P., 10th Floor, One Landmark Square, Stamford, CT, 06901-2682, US

(73) Assignee: Avon Products, Inc.

(21) Appl. No.: 11/319,947

(22) Filed: Dec. 28, 2005

(51) Int. Cl. A61K 8/19

(52) U.S. Cl. 424/63

(57) ABSTRACT

There is an anhydrous topical composition. The composition has a) an active ingredient; b) a base compound of a carbonate or bicarbonate salt of an alkali metal or an alkaline earth metal; c) an acid compound; and d) a cosmetically acceptable anhydrous vehicle. The base compound and the acid compound are reactable and are present in amounts sufficient to foam the topical composition when the topical composition is exposed to water. The topical composition changes color upon exposure to water. There is also a method for imparting a cosmetic effect to a topical surface.

### BACKGROUND OF THE INVENTION

1. Field of the Invention

The present invention relates to an anhydrous topical composition that changes color and foams upon contact with water. The present invention further relates to a facial mask composition that changes color and foams upon contact with water. The present invention also relates to a method for imparting a cosmetic effect to a topical surface of the human body.

2. Description of the Related Art

Cosmetic compositions are commonly formulated to be aesthetically pleasing to the user. Colorants and/or pigments are commonly added to such compositions to improve their appearance. Cosmetic compositions are also sometimes formulated to change colors during application and/or use.

Cosmetic compositions are sometimes formulated to provide a phase change during application or use. Examples of such change are liquid to solid (and vice versa), liquid to foam (and vice versa), or semi-solid to foam (and vice versa).

Accordingly, it would be desirable to have a topical composition that provided a color change during use. Further, it would be desirable to have a topical composition that changed phase during use.

### SUMMARY OF THE INVENTION

An object of the present invention is to provide an anhydrous topical composition that changes color upon contact with water.

Another object of the present invention is to provide an anhydrous topical composition that foams upon contact with water.

Still another object of the present invention is to provide an anhydrous facial mask composition that changes color and foams upon contact with water.

Still yet another object of the present invention is to provide a method for imparting a cosmetic effect to a topical surface of the human body.

According to this and other objects of the invention, there is an anhydrous topical composition. The composition has a) an active ingredient; b) a base compound of a carbonate or bicarbonate salt of an alkali metal or an alkaline earth metal; c) an acid compound; and d) a cosmetically acceptable anhydrous vehicle. The base compound and the acid compound are reactable and are present in amounts sufficient to foam the topical composition when the topical composition is exposed to water.

In another aspect of the invention the composition further contains a colorant to provide a predetermined color to the composition. The topical composition changes color and foams upon exposure to water.

According to this and other objects of the invention, there is a method for imparting a cosmetic effect to a topical surface. The method has the steps of a) applying the anhydrous composition described above to the topical surface of the human body, b) contacting the anhydrous composition with water to allow it to foam to form a foamed topical composition; and c) removing the foamed topical composition from the topical surface.

### DETAILED DESCRIPTION OF THE INVENTION

It was surprisingly found that there is an anhydrous topical composition that provided a color change during application and use. It was further surprisingly found that there is an anhydrous topical composition that both foams and provides a color change. It was still further surprisingly found that there is an anhydrous facial mask composition that both foams and provides a color change. The products of the present invention are also appropriate for use as a foot mask.

The anhydrous topical composition surprisingly and unexpectedly exhibits a color change upon contact with or exposure to water. While any color is suitable, the initial color of the composition is usually a mud or dark color, such as brown, gray, blue, or black. Such dark color is preferred, as it permits the user to determine where the composition has been applied. After contact with or exposure to water, the composition changes to a lighter color, such as white or beige. White is a preferred final color. The color change is aesthetically appealing to the product user and signals a later phase in the application/use cycle of the composition. The initial color should be different than the final or end use color.

The anhydrous topical composition also foams upon contact with or exposure to water. The water provides an aqueous environment necessary to effect a neutralization reaction between the acid compound and the base compound. The reaction produces carbon dioxide, which mingles with the anhydrous topical composition to form a foamed topical composition. Further during the reaction, which is exothermic, heat is generated, which helps to clean the topical surface of the human body to which the composition is applied. The generated heat is particularly useful in cleaning skin pores. The phase change is aesthetically appealing to the product user and signals a later phase in the application/use cycle of the composition.

The topical composition is substantially anhydrous in that the substantial absence of water is critical to prevent premature reaction of the acid compound and the base compound. Prevention of premature reaction prevents premature color change and foaming. To prevent premature reaction and loss of reactants, the compositions of the present invention may also contain a small amount of a deliquescent agent, for example, calcium chloride, fumed silica or zinc chloride. Typically, the amount of deliquescent agent is about 2% or less by total weight of the composition.

As referred to herein, the term “topical surface” refers to the exterior surface of the epidermis (the skin). Topical surface includes the exterior surface of any portion of the human body.

The acid compound employed in the topical composition can be any organic or inorganic acid suitable for cosmetic and/or topical use. Organic acids are preferred. Suitable organic acids include, but are not limited to, citric acid, tartaric acid, ascorbic acid, succinic acid, malic acid, malonic acid, fumaric acid, glutaric acid, adipic acid, pimelic acid, maleic acid, phthalic acid, glutamic acid, aspartic acid, glycolic acid, tartronic acid, hydroxybenzoic acid, salicylic acid, tropic acid, cinnamic acid, phenylacetic acid, nicotinic acid, sorbic acid, trimellitic acid, polyacrylic acid, polyaspartic acid, and combinations thereof. Citric acid is preferred. Boric acid is a preferred inorganic acid, although phosphoric and mineral acids are useful as well.

The base compound employed in the topical composition is a carbonate or bicarbonate salt of an alkali metal or an alkaline earth metal. Suitable carbonate/bicarbonate compounds include, but are not limited to, sodium carbonate, sodium bicarbonate, potassium carbonate, potassium bicarbonate, magnesium carbonate, magnesium bicarbonate, calcium carbonate, calcium bicarbonate, and combinations thereof.

Preferably, the base compound is encapsulated. A preferred encapsulated base compound is an encapsulated alkali metal bicarbonate powders, such as those of sodium bicarbonate and potassium bicarbonate. The powders will vary in particle size and shape and be encapsulated with any of a multitude of known encapsulants, such as hydrogenated vegetable oil, sunflower oil, palm oil, monoglycerides, diglycerides, and polyethylene glycol. Similarly, a preferred acid compound is an encapsulated acid. The particles will have a size range distribution and be encapsulated with any known encapsulant, including those disclosed above for the base compound, and further including monosodium citrate. There are several ways of coating available for both acid and base, and they serve the same purpose of preventing premature undesired reactions.

When mixed with water, bicarbonates and carbonates form carbonic acid and salts. The carbonic acid is unstable in water and breaks down rapidly to form CO₂ and water, per the following scheme: 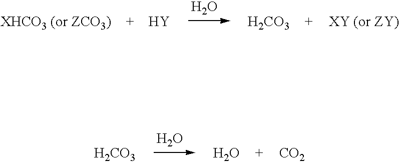
wherein

X is an alkali metal cation or NH₄⁺,

Z is an alkaline earth metal cation,

HY is a mineral or organic acid,

and

XY is alkali or alkaline earth metal salt.

The acid compound and the base compound may be employed in even or uneven stoichiometric proportions. If desired, the two compounds may be employed in uneven stoichiometric proportions to ensure that one compound will be substantially consumed (converted) during the reaction. The base compound will be present in excess to ensure substantially complete reaction of the acid compound. However, it is also possible to formulate the products so that the acid compound is in excess. This will result in having unreacted acid following foaming. Accordingly, the composition following the addition of water, i.e., on the body of the consumer, will have a somewhat acid pH, which would exhibit a mild exfoliating effect on the skin. Depending on whether the acid component or the base component is in excess, such final in use end product pH values may range from about 3.5 to about 8.5, preferably not less than 3.8 and not greater than 8.0, and most preferably from about 4 to about 7.5. A typical formal ratio (equivalents acid/equivalents base) is from about 1:10 to about 10:1, preferably from about 1:5 to about 5:1. For better foaming and a somewhat alkaline in use end product pH value, a particularly preferred formal ratio is from about 1:1 to about 1:3. When the product compositions will be formulated to provide an acidic in use end product pH value, the formal ratio (equivalents acid/equivalents base) is most preferably from about 3:1 to about 1:1.

The acid compound and the base compound are present in amounts sufficient to foam the anhydrous composition and change the color thereof when the anhydrous composition is exposed to water. The acid compound is preferably present at about 1 to about 30 wt % and most preferably present at about 3 to about 20 wt % based on the total weight of the topical composition. The base compound is preferably present at about 1 to about 30 wt % and most preferably present at about 3 to about 20 wt % based on the total weight of the topical composition.

The relative amounts of acid and base present in the composition will vary depending on the identity of each and their valances. Thus, when citric acid is used, which has three carboxyl groups available for reaction, the composition contains, for theoretical complete reaction, a molar amount that is one third the molar amount of sodium bicarbonate.

The active ingredient can take the form of any compound that imparts a cosmetic, functional, and/or medicinal effect to a topical surface. Useful active ingredients include, but are not limited to, anesthetics, anti-allergenics, antifungals, antimicrobials, anti-inflammatories, antiseptics, astringents, barrier agents, botanical extracts, chelating agents, depigmenting agents, detergents/cleansers, emollients, heat forming compounds, exfoliants, film formers, firming agents, fragrances, humectants, insect repellents, lubricants, moisturizers, pharmaceutical agents, preservatives, skin protectants, skin penetration enhancers, stabilizers, sunscreen agents, surfactants, vitamins, and any combinations thereof.

Particularly preferred active ingredients include exfoliants, moisturizers, heat forming compounds, and humectants.

The anhydrous composition has an active ingredient in an amount sufficient to impart a cosmetic effect to the topical surface. The amount will vary considerably depending on the type of cosmetic effect desired and the properties of the active ingredient. The active ingredient will typically be present at about 0.001 to about 30 wt % and more typically present at about 0.01 to about 15 wt %, and most typically present at about 0.1 to about 5 wt % based on the total weight of the topical composition.

The present application may have one or more colorants/pigments as desired to modify the appearance of the anhydrous composition (initial) or the foamed composition (final). Examples of useful pigments/colorants include, but are not limited to, mica, iron oxides. Other color additives resulting from the carefully controlled heat treatment of food-grade carbohydrates may be used.

Useful cosmetically acceptable vehicles include non-aqueous fluids, such as polyols, alkyl benzoates, esters, silicone fluids, hydrocarbon oils, triglycerides, ethers, fatty alcohols, fatty acid fluids, and low molecular weight alcohol having 2 to 8 carbons such as isopropyl alcohol.

It is possible for an ingredient to serve more than one function in the anhydrous composition. For instance, an active ingredient can also function as a cosmetically acceptable vehicle. Glycerol and propylene glycol, for example, can serve as humectants (active ingredient) and as vehicles.

The anhydrous topical composition takes a liquid or semisolid form. The composition is fluidic enough to be spread by hand, applicator, or implement. Useful forms include solution, lotion, paste, cream, gel, and pomade. Composition fluid properties can be modified as desired by the addition of adjuvants such as thickeners, viscosity modifiers, and the like.

The anhydrous composition can be applied to any topical surface, exposed to water, and then removed to impart a desired cosmetic effect thereto. Useful topical surfaces include skin. Any area of the skin, such as the hands and face, may be treated. The composition may be applied as often and/or as long as needed or desired. For instance, the composition may be applied one or more times per day for as little as a day, a week or more, or two weeks or more.

The anhydrous composition is useful in any type of cosmetic product. Suitable compositions include facial mask, skin cleanser, and skin exfoliant. A preferred composition is a facial mask composition.

Facial mask compositions can be spread over the surface of the skin of the face. The facial mask composition is then allowed to remain on the face for a period of time, typically about 1 to about 15 minutes and preferably about 5 to about 10 minutes, to give time for one or more active ingredients to impart a cosmetic effect or benefit. The facial mask composition preferably has a mud or dark color, such as brown, gray, blue, or black. The dark color provides a visual background to facilitate uniform application over the topical surface. Then water is applied to the face. Water may be dripped on the face, splashed, or applied by means of a wet towelette. The water causes the acid compound and the base compound to react to form carbon dioxide and a neutralized salt. The evolution of carbon dioxide causes the facial mask composition to foam, evolve heat, and turn a different color, typically white. The foam generation as well as the color change provide a signal that a sufficient amount of water has been applied and the product is working. The foamed mask composition may then, if desired, be left on the face for a period of time, typically about 1 to about 15 minutes, preferably about 5 to about 10 minutes, to give time for the one or more active ingredients to impart a cosmetic effect(s) or benefit(s) or to permit cleansing of skin pores. The foamed mask composition is then removed, i.e., rinsed or washed off.

Preferred face mask compositions preferably have humectants, moisturizers, heat forming compounds, and thickeners. More preferred thickeners include clays, such as kaolin, hectorite, bentonite, and montmorillonite. Useful humectants include glycerol and polyols, such as ethylene glycol, propylene glycol, pentylene glycol, and hexylene glycol.

Heat forming compounds are compounds that liberate heat upon dissolution in water. These compounds are included in the composition as a dry powder. The liberation of heat when water is applied to the film of the anhydrous composition augments the heat occasioned by the reaction of the acid and base. Suitable heat forming compounds include zeolites, PPG-12 buteth-16 sold as UCON 50-HB-660 by Amerchol, magnesium sulfate (MgSO₄), calcium chloride, and other material(s) that are exothermic_and have a negative heat of solution. These compounds may be incorporated in the anhydrous composition in an amount of from about 1 to about 80 percent by total weight of the composition, preferably from about about 1 to about 25 percent. Glycols are also known to provide a heating effect, and can be incorporated into the compositions as part of the gelling system, discussed below.

In a preferred embodiment, the anhydrous composition is in the form of a gel. The gel system has a gelling agent and a polyol. The gelling agent is present in the anhydrous composition in an amount of from about 1 to about 30 percent by weight of the total composition.

The gelling agent is preferably a clay material, especially a clay selected from the group consisting of kaolin, bentonite, and hectorite. A preferred clay is kaolin. Clay is present in the anhydrous composition in an amount of from about 1 to about 15 percent by total weight of the composition.

Suitable polyols have 3 to 10 carbons. Preferred polyols are butylene glycol, hexylene glycol, dipropylene glycol, and glycerin. The polyols are present in the anhydrous composition in an amount of up to about 80% by weight, preferably from about 10 to about 40 percent by weight to the total composition.

The composition preferably exhibits a thixotropic rheology, The composition has a viscosity sufficient to permit application to the body of the consumer and which avoids dripping or running from the body regardless of spatial orientation. Typically, the composition has a viscosity of about 500 to 1,000,000 cps. The composition preferably exhibits a viscosity of about 10,000 to about 500,000 cps at 25° C., and most preferably from about 10,000 to 200,000 cps, as measured by a Brookfield viscometer having an appropriate spindle for the viscosity region. This viscosity level of the thixotropic composition allows the user to apply the composition to the face or other body surface without having the composition drip off. Additionally, this viscosity level is beneficial in that it prevents excessive dripping of the wetted composition. This viscosity level also does not prevent reaction of the acid compound and the base compound.

The composition may be stored in any suitable container. Preferably, however, the composition is sold in an airtight, single-use pouch to prevent moisture from prematurely causing reaction. As previously discussed, a small amount of a deliquescent material can be incorporated in the composition to absorb any moisture that may be present. Suitable materials are zinc chloride, calcium chloride, and silica.

Unless otherwise indicated below, all parts, percentages, and proportions are by weight.

### EXAMPLES

The following composition is suitable for use as a facial mask. The composition has the ingredients set forth in the Table below.

TABLE

| Components | Percent |
|---|---|
| Butylene Glycol | 16.0 |
| Hexylene Glycol | 11.4 |
| Dipropylene Glycol | 7.0 |
| Polyethylene Glycol 400 | 3.8 |
| Hydroxyethyl Cellulose | 0.5 |
| Dimethicone | 0.5 |
| PEG-20 Methyl Glucose | 1.7 |
| Sesquistearate | |
| Xanthan Gum | 1.5 |
| Kaolin | 12.0 |
| Magnesium Sulfate-Anhydrous | 20.0 |
| Sodium Bicarbonate | 18.6 |
| Citric Acid | 6.2 |
| KTZ pearl or other color | 1.5 |
| additive | |
| Peppermint fragrance | 0.1 |
| Paraben Esters-Liquid Blend | 0.7 |
| | 100.0 |

The composition is made according to the following paragraphs. All equipment used in processing, storage, and filling must be moisture-free.

Add the butylene glycol, dipropylene glycol and hexylene glycol into a mill or mixer. Begin mixing and slowly add the hydroxyethylcellulose. Heat contents of mixer to 165-170° F. (74-77° C.). Mill and mix rapidly until a uniform dispersion is formed.

Add the polyethylene glycol, the PEG-20 Methyl Glucose Sesquistearate, paraben esters and dimethicone into a side kettle. Heat to 165-170° F. (74-77° C.) and mix until uniform. Slowly add the xanthan gum into the side kettle. Mix until uniform and transfer contents from side kettle into mixer. Mill and mix rapidly with blade until a uniform dispersion is formed. Maintain temperature at 165-170° F. (74-77° C.). Add the dipropylene glycol into the side kettle for flush/rinse purposes with mixing and transfer into mixer, thereby flushing the side kettle, pump and lines. Mill and mix with blade for 5 minutes. At the conclusion of the mixing period, slowly add kaolin into mixer while maintaining the temperature at 165-170° F. (74-77° C.). Cool the contents of mixer to 105-110° F. (40-43° C.). Sequentially add magnesium sulfate, sodium bicarbonate and citric acid into mixer and mill. Mix the pigment/pearlescent into the mill. Cool the contents of the mixer to 85-90° F. (29-32° C.). Add peppermint oil into the mixer. Mix until uniform and cool to room temperature.

The composition is in the form of an anhydrous gel having a thixotropic rheology. The composition is anhydrous and must be kept dry until use.

The composition is applied to the face using an applicator stick to provide a uniform coating on the face. The composition is left on the face for five minutes, and then wetted with water using a partially saturated washcloth. Upon contact with water, the composition foams. Additionally, the temperature of the product on the face also increases. After five minutes the composition is removed from the face by thoroughly rinsing the face with water. The face is cleaner and has a healthy glow.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A topical composition, comprising:
a) an active ingredient in an amount sufficient to impart a cosmetic effect;
b) a base compound of a carbonate or bicarbonate salt of an alkali metal or an alkaline earth metal;
c) an acid compound; and
d) a cosmetically acceptable anhydrous vehicle,
wherein the base compound and the acid compound are reactable and are present in amounts sufficient to foam the composition when the composition is exposed to water, wherein the composition changes color upon exposure to water, wherein the composition is substantially anhydrous.

2. The composition of claim 1, wherein the base compound is present at about 1 to about 30 wt % based on the total weight of the topical composition.

3. The composition of claim 1, wherein the acid compound is present at about 1 to about 30 wt % based on the total weight of the topical composition.

4. The composition of claim 1, wherein the base compound is selected from the group consisting of sodium carbonate, sodium bicarbonate, potassium carbonate, potassium bicarbonate, magnesium carbonate, magnesium bicarbonate, calcium carbonate, calcium bicarbonate, and combinations thereof.

5. The composition of claim 1, wherein the acid compound is selected from the group consisting of citric acid, tartaric acid, ascorbic acid, succinic acid, malic acid, malonic acid, fumaric acid, glutaric acid, adipic acid, pimelic acid, maleic acid, phthalic acid, glutamic acid, aspartic acid, glycolic acid, tartronic acid, hydroxybenzoic acid, salicylic acid, tropic acid, cinnamic acid, phenylacetic acid, nicotinic acid, sorbic acid, trimellitic acid, and combinations thereof.

6. The composition of claim 1, wherein the formal ratio of the acid compound to the base compound is from about 10:1 to about 1:10.

7. The composition of claim 1, wherein the active ingredient is selected from the group consisting of anesthetics, anti-allergenics, antifungals, antimicrobials, anti-inflammatories, antiseptics, barrier agents, botanical extracts, chelating agents, colorants, depigmenting agents, detergents, emollients, exfoliants, film formers, fragrances, humectants, heat forming compounds, insect repellents, lubricants, moisturizers, pharmaceutical agents, preservatives, skin protectants, skin penetration enhancers, stabilizers, sunscreen agents, surfactants, vitamins, and any combinations thereof.

8. The composition of claim 1, further comprising a heat forming compound is selected from the group consisting of magnesium sulfate, calcium chloride, PPG-12 buteth-16, zeolites, and combinations thereof and is present in an amount from about 1 to about 25 percent by weight of the total composition.

9. The composition of claim 1, wherein the composition is a facial mask composition.

10. A method for imparting a cosmetic effect to a topical surface of a human body, comprising:
a) applying to the topical surface an anhydrous composition, the anhydrous composition having
i) an active ingredient in an amount sufficient to impart a cosmetic effect;
ii) a base compound of a carbonate or bicarbonate salt of an alkali metal or an alkaline earth metal;
iii) an acid compound; and
iv) a cosmetically acceptable anhydrous vehicle;
wherein the base compound and the acid compound are reactable and are present in amounts sufficient to foam the anhydrous composition when the anhydrous composition is exposed to water;
b) contacting the anhydrous composition with water to allow it to foam to form a foamed composition; and
c) removing the foamed composition from the topical surface.

11. The method of claim 10, wherein the anhydrous composition is left for a period of time prior to contacting it with water, and wherein the foamed topical composition is left for a period of time prior to removing it from the topical surface.

12. The method of claim 10, wherein the topical surface is a face.

13. The method of claim 10, wherein the topical surface is a foot.

14. The method of claim 10, wherein the composition is a gel.

15. The method of claim 14, wherein the gel has a gelling system having a clay and a polyol.

16. The method of claim 10, wherein the composition has a thixotropic rheology having a viscosity of about 25,000 to about 500,000 cps. at 25° C.

17. The method of claim 10, wherein the pH of the composition following step (b) is from about 3.5 to about 8.5.

18. The method of claim 10, wherein the composition further comprises a heat generating compound.
